(19) United States
(12) Patent Application Publication
(10) Pub. No.: US 2018/0319571 A1
(43) Pub. Date: Nov. 8, 2018

(54) Garbage Bag Dispensing Assembly

(71) Applicant: John Villarreal, La Puente, CA (US)

(72) Inventor: John Villarreal, La Puente, CA (US)

(21) Appl. No.: 15/589,549

(22) Filed: May 8, 2017

(51) Int. Cl. B65D 83/08; B65F 1/00; B65D 43/16; B65D 25/54; B65D 25/04; B65D 43/22; B65D 55/02; A61B 42/40

(52) U.S. Cl. CPC B65D 83/0805; B65F 1/0006; B65D 43/163; A61B 42/40; B65D 25/04; B65D 43/22; B65D 55/02; B65D 25/54

(57) ABSTRACT

A garbage bag dispensing assembly includes a box that is coupled to a support surface thereby facilitating the box to be manipulated. A plurality of rolls of garbage bags is selectively positioned in the box. The box has a plurality of slots extending into an interior of the box and each of the slots facilitates the garbage bags to be dispensed outwardly therefrom. A box of gloves is selectively positioned in the box. The box has a window extending into an interior of the box to facilitate access to the box of gloves. a door is hingedly coupled to the box and the door selectively opens and closes the box.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to dispensing devices and more particularly pertains to a new dispensing device for storing and dispensing garbage bags and plastic gloves.

### BRIEF SUMMARY OF THE INVENTION

An embodiment of the disclosure meets the needs presented above by generally comprising a box that is coupled to a support surface thereby facilitating the box to be manipulated. A plurality of rolls of garbage bags is selectively positioned in the box. The box has a plurality of slots extending into an interior of the box and each of the slots facilitates the garbage bags to be dispensed outwardly therefrom. A box of gloves is selectively positioned in the box. The box has a window extending into an interior of the box to facilitate access to the box of gloves. a door is hingedly coupled to the box and the door selectively opens and closes the box.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a garbage bag dispensing assembly according to an embodiment of the disclosure.

FIG. 2 is a perspective view of an embodiment of the disclosure.

FIG. 3 is a back view of an embodiment of the disclosure.

FIG. 4 is a cross sectional view taken along line 4-4 of FIG. 3 of an embodiment of the disclosure.

FIG. 5 is a phantom perspective view of an embodiment of the disclosure.

### DETAILED DESCRIPTION OF THE INVENTION

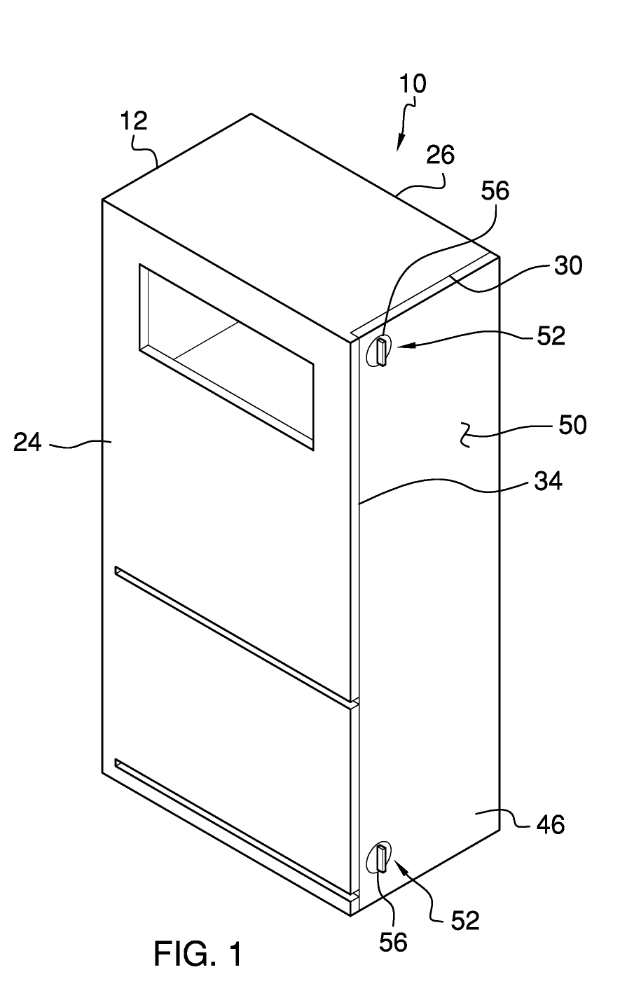

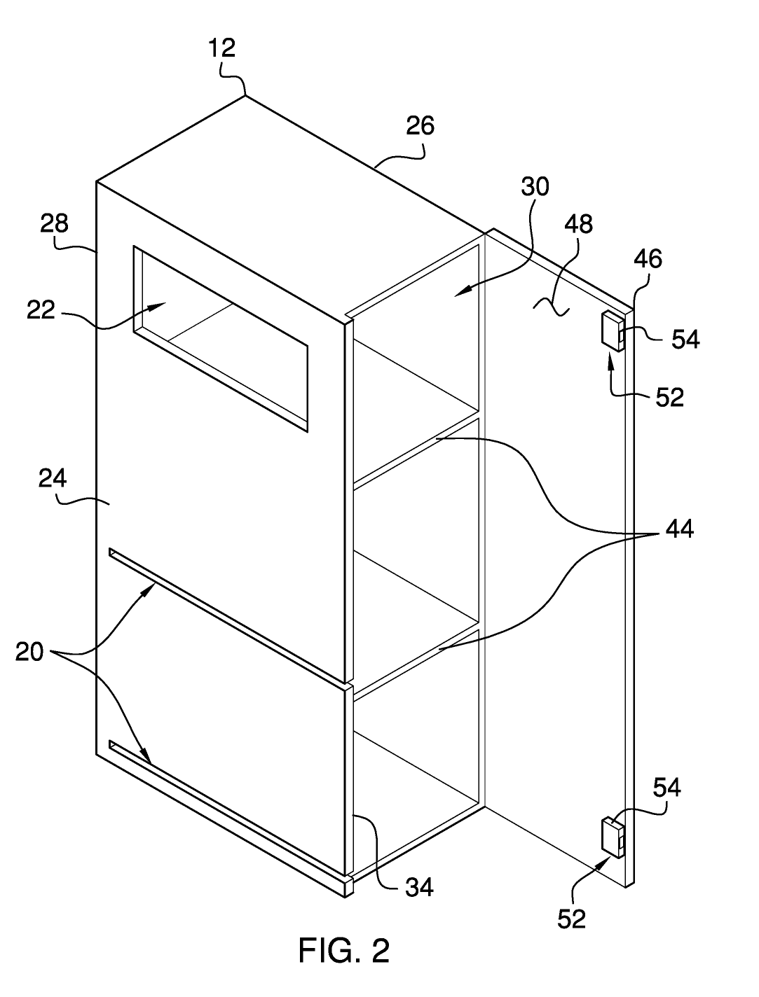

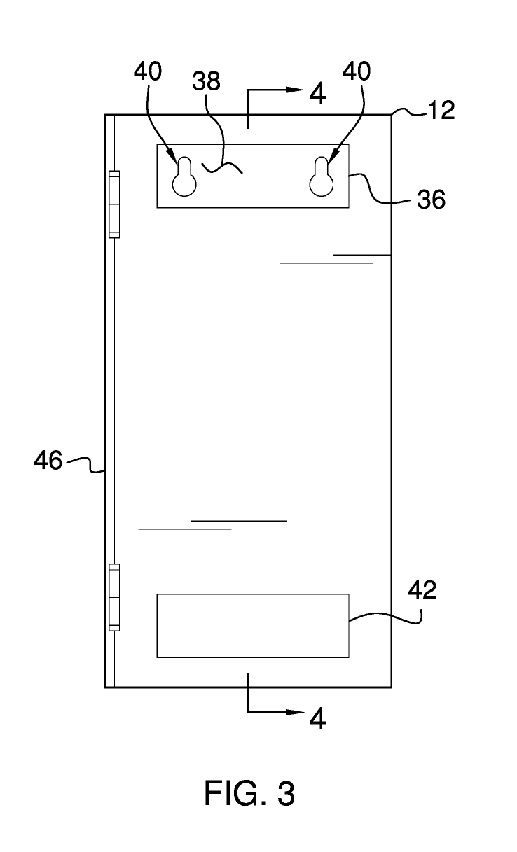

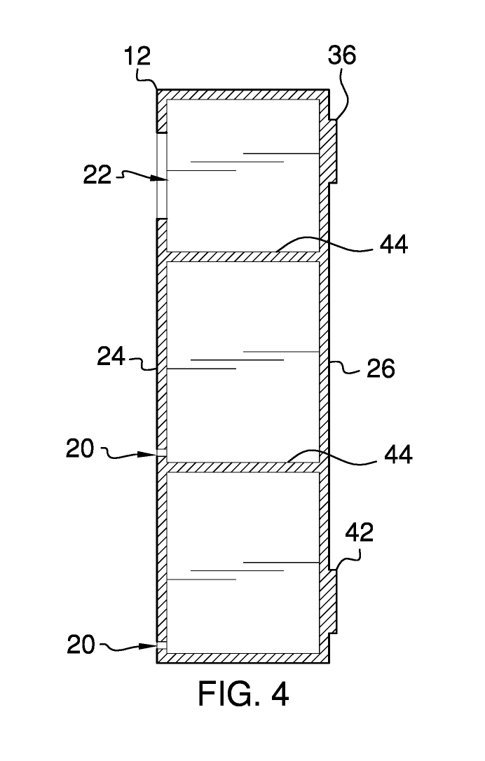

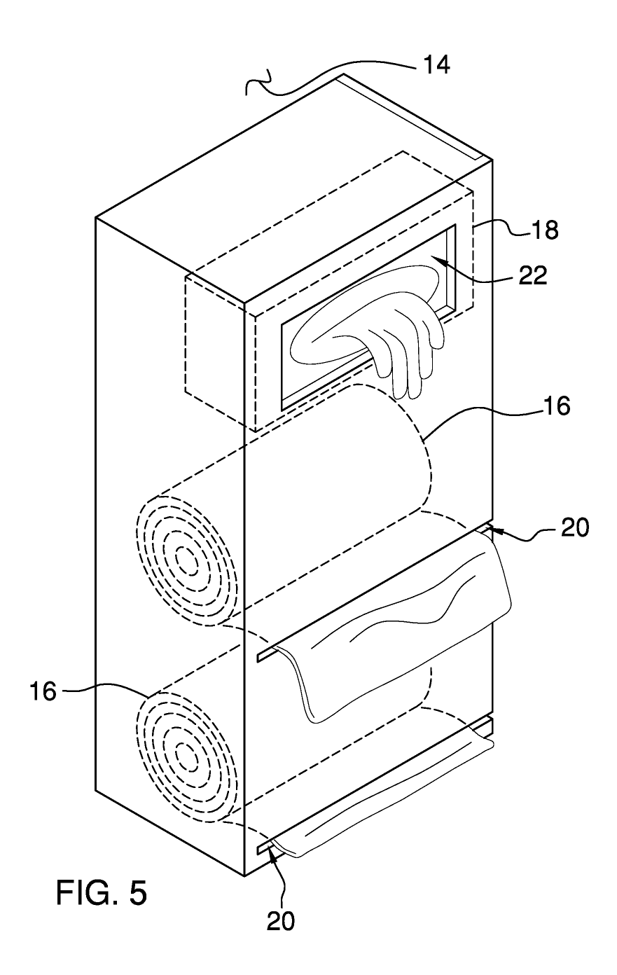

With reference now to the drawings, and in particular to FIGS. 1 through 5 thereof, a new dispensing device embodying the principles and concepts of an embodiment of the disclosure and generally designated by the reference numeral 10 will be described.

As best illustrated in FIGS. 1 through 5, the garbage bag dispensing assembly 10 generally comprises a box 12 that is coupled to a support surface 14 thereby facilitating the box 12 to be manipulated. The support surface 14 may be a wall or other vertical support surface 14. A plurality of rolls of garbage bags 16 is positioned in the box 12 and the garbage bags 16 may be plastic garbage bags 16 or the like. The box 12 has a plurality of slots 20 extending into an interior of the box 12 to facilitate the garbage bags 16 to be dispensed outwardly therefrom. Additionally, a box of gloves 18 is positioned in the box 12 and the box of gloves 18 may be latex gloves or the like. The box 12 has a window 22 extending into an interior of the box 12 to facilitate access to the box of gloves 18.

The box 12 has a front wall 24, a rear wall 26, a first lateral wall 28, a second lateral side 30 and an inside surface 32. The second lateral side 30 is open to access the interior of the box 12 and each of the slots 20 extends through the front wall 24. Each of the slots 20 is horizontally oriented and extends from the second lateral side 30 toward the first lateral wall 28. The slots 20 are spaced apart from each other on the front wall 24. The window 22 extends through the front wall 24 and the window 22 is spaced from the pair of slots 20. Additionally, the front wall 24 extends beyond the second lateral side 30 to define a lip 34 on the box 12.

A first block 36 is coupled to the rear wall 26 of the box 12 and the first block 36 has a distal surface 38 with respect to the rear wall 26. The distal surface 38 has a pair of slots 40 extending toward the rear wall 26 and each of the slots 40 receives a support, such as a nail or a screw, to suspend the box 12 on the support surface 14. A second block 42 is coupled to the rear wall 26 of the box 12 and the second block 42 is spaced from the first block 36. The second block 42 abuts the support surface 14 to space the box 12 from the support surface 14.

A plurality of shelves 44 is provided and each of the shelves 44 is positioned within the box 12. Each of the garbage bags 16 and the box of gloves 18 is positioned a selected one of the shelves 44. Each of the shelves 44 extends between the rear wall 26 and the front wall 24 and each of the shelves 44 is aligned with an associated one of the slots 20 and the window 22. A door 46 is hingedly coupled to the box 12 such that the door 46 selectively opens and closes the box 12. The door 46 has a first surface 48 and a second surface 50 and the door 46 is positioned on the second lateral side of the box 12. The door 46 is positioned in an open position to expose the second lateral side 30 and the door 46 is positioned in a closed position to cover the second lateral side 30. Additionally, the second surface 50 is aligned with the lip 34 when the door 46 is closed.

A pair of latches 52 is provided and each of the latches 52 is coupled to the door 46. Each of the latches 52 engages the box 12 when the door 46 is closed to retain the door 46 in the closed position. Each of the latches 52 comprises a lock 54 that is coupled to the first surface 48 of the door 46. The lock 54 engages the inside surface 32 corresponding to the front wall 24 of the box 12 when the door 46 is closed to retain the door 46 in the closed position. The lock 54 may be a mechanically actuated door 46 lock 54 of any conventional design.

A knob 56 is rotatably coupled to the second surface 50 of the door 46 and the knob 56 is selectively manipulated. The knob 56 extends through the door 46 and is mechanically coupled to the lock 54. The knob 56 is selectively rotated in a first direction and a second direction. The lock 54 disengages from the door 46 when the knob 56 is rotated in the first direction thereby facilitating the door 46 to be opened. Additionally, the lock 54 engages the door 46 when the knob 56 is rotated in the second direction thereby locking the door 46 in the closed position.

In use, the box 12 is suspended on the support surface 14 and each of the rolls of garbage bags 16 and the box of gloves 18 is positioned on a selected one of the shelves 44. The garbage bags 16 are extended through an associated one of the slots 20 and the door 46 is closed. The knob 56 corresponding to each of the latches 52 is manipulated to lock 54 the door 46 in the closed position. A garbage bag 16 is drawn outwardly through a selected one of the slots 20 for use for cleaning or the like. Additionally, a selected number of the gloves 18 are drawn through the window 22. In this way the garbage bags 16 and gloves 18 are accessible and organized. Each of the rolls of garbage bags 16 and the box of gloves 18 are replaced when depleted.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. (canceled)

2. (canceled)

3. (canceled)

4. (canceled)

5. (canceled)

6. (canceled)

7. (canceled)

8. A garbage bag dispensing assembly being configured to contain and selectively dispense garbage bags, said assembly comprising:
a box being configured to be coupled to a support surface thereby facilitating said box to be manipulated, said box being configured to have a plurality of rolls of garbage bags positioned therein, said box having a plurality of slots extending into an interior of said box wherein each of said slots is configured to facilitate the garbage bags to be dispensed outwardly therefrom, said box being configured to have a box of gloves positioned therein, said box having a window extending into an interior of said box wherein said window is configured to facilitate access to the box of gloves, said box having a front wall, a rear wall, a first lateral wall, a second lateral side, and an inside surface, said second lateral side being open to access said interior of said box, each of said slots extending through said front wall, each of said slots being horizontally oriented and extending from said second lateral side toward said first lateral wall, said slots being spaced apart from each other on said front wall, said window extending through said front wall, said window being spaced from said pair of slots, said front wall extending beyond said second lateral side to define a lip on said box;
a plurality of shelves, each of said shelves being positioned within said box wherein each of said shelves is configured to have selected ones of the garbage bags and the box of gloves positioned thereon, each of said shelves extending between said back wall and said front wall wherein said shelves divide said interior of said box into a plurality of compartments, each of said slots being adjacent to an associated one of said compartments, each of said slots being positioned adjacent to a bottom of said associated compartment;
a door being hingedly coupled to said box such that said door selectively opens and closes said box, said door having a first surface and a second surface, said door being positioned on said second lateral side of said box, said door being positioned in an open position to expose said second lateral side, said door being positioned in a closed position to cover said second lateral side, said second surface being aligned with said lip when said door is closed; and
a pair of latches, each of said latches being coupled to said door wherein each of said latches is configured to be manipulated, each of said latches engaging said box when said door is closed to retain said door in said closed position, each of said latches comprising:
a lock being coupled to said first surface of said door, said lock engaging said inside surface corresponding to said front wall of said box when said door is closed to retain said door in said closed position, and
a knob being rotatably coupled to said second surface of said door wherein said knob is configured to be manipulated, said knob extending through said door and being mechanically coupled to said lock, said knob being selectively rotated in a first direction and a second direction, said lock disengaging from said door when said knob is rotated in said first direction thereby facilitating said door to be opened, said lock engaging said door when said knob is rotated in said second direction thereby locking said door in said closed position.
